(19) United States
(12) Patent Application Publication
(10) Pub. No.: US 2022/0062138 A1
(43) Pub. Date: Mar. 3, 2022

(54) COSMETIC COMPOSITION COMPRISING HYDROSOLUBLE UV FILTERS

(71) Applicant: L'OREAL, Paris (FR)

(72) Inventors: Laure ADER, Chevilly La Rue (FR); Flavie GILLANT, Chevilly La Rue (FR); Angelina ROUDOT, Chevilly La Rue (FR)

(21) Appl. No.: 17/416,568

(22) PCT Filed: Dec. 26, 2019
(86) PCT No.: PCT/EP2019/087041

§ 371 Date: Jun. 21, 2021

(30) Foreign Application Priority Data
Dec. 26, 2018 (FR) 18 74154

(51) Int. Cl. A61K 8/46; A61K 8/34; A61K 8/81; A61K 8/73; A61K 8/44; A61Q 17/04

(52) U.S. Cl. CPC A61K 8/466; A61K 8/345; A61K 8/8147; A61K 8/73; A61K 2800/522; A61Q 17/04; A61K 2800/48; A61K 2800/594; A61K 2800/544; A61K 8/445

(57) ABSTRACT

The present invention relates to a composition, particularly a cosmetic composition, comprising the following in a physiologically acceptable aqueous medium: at least one filter chosen from among hydrosoluble filters capable of absorbing UV from 320 to 400 nm (UVA), hydrosoluble filters capable of absorbing UV from 280 to 320 nm (UVB) and mixtures thereof. a first thickening agent chosen from among carrageenans; and at least one second thickening agent chosen from among synthetic anionic thickening polymers, branched carboxylated thickening polysaccharides and mixtures thereof.

This invention relates to new compositions, and particularly cosmetics comprising at least one filter chosen from among hydrosoluble filters capable of absorbing UV from 320 to 400 nm (UVA), hydrosoluble filters capable of absorbing UV from 280 to 320 nm (UVB) and mixtures of them, and an association of specific thickening agents, in a physiologically acceptable aqueous medium.

It is known that radiation with wavelengths between 280 nm and 400 nm enables tanning of the human epidermis, while radiation with wavelengths between 280 and 320 nm, known as UVB rays, impede the development of a natural tan. Exposure can also lead to a prejudicial change in the biomechanical properties of the epidermis, resulting in the appearance of wrinkles leading to premature aging of the skin.

It is also known that UVA rays with a wavelength of between 320 and 400 nm penetrate into the skin into the skin more deeply than UVB rays. UVA rays cause immediate and persistent tanning of the skin. Daily exposure to UVA rays, even short duration, under normal conditions, can damage collagen and elastin fibers, which results in a modification to the microrelief of the skin, the appearance of wrinkles and unequal pigmentation (spots and uneven color).

A wide variety of photoprotective compositions to protect keratin materials and particularly the skin, against the harmful effects induced by UVA and/or UVB radiation, are already known in prior art. Essentially, they contain a combination of several organic or inorganic UV filters, transferred in the oily phase and/or the aqueous phase as an anti-UV active agent and are generally offered in emulsion or gel type galenical form.

It is also known that high filter ratios are necessary to reach high level filter efficiencies.

However, high ratios with UV filters do not facilitate the production of compositions that have a stable and pleasing texture.

Thus, formulations with a high filter capacity generally have uncomfortable or even unpleasant sensorial aspects concealing the freshness and comfort of formulas. In particular, the weak point of photoprotective formulations with a high protection index is often a significantly greasy sensation, and therefore lack of lightweight feeling of the textures obtained, but also a white appearance in application and therefore not invisible on the skin.

Furthermore, the introduction of UV filters with a high content usually causes destabilization problems. This instability can even sometimes cause a phase change in the emulsion and/or a loss of viscosity of the composition, making the formulation inefficient or even unusable.

Aqueous dosages have already been considered to mitigate the above-mentioned undesirable effects, and in particular to obtain a fresh effect on application while being invisible on the skin. However, these aqueous compositions containing UV filters are generally sticky and therefore uncomfortable.

Consequently, there is a need for an aqueous photoprotective composition with a high level of UV protection that is perfectly stable and homogeneous, in other words that is unaffected by demixing phenomena.

There is also a need for an aqueous photoprotective composition with a high level of UV protection and that is also transparent. In particular, there is a need for a photoprotective composition that is transparent even after application on the skin.

There is also a need for an aqueous photoprotective composition with a high level of UV protection, that has good cosmetic properties on application, particular that is easy to apply, without a greasy or sticky finish on the skin, particularly after drying, and with a pleasant sensation, materialized particularly by easy sliding during application.

The purpose of this invention is to offer new cosmetic compositions providing a solution to these problems.

In particular, the purpose of this invention is to offer new aqueous compositions, particularly cosmetic, comprising hydrosoluble UV filters, that are stable and homogeneous.

The present invention thus relates to a composition, particularly a cosmetic composition, comprising, in a physiologically acceptable aqueous medium:

at least one filter chosen from among hydrosoluble filters capable of absorbing UV from 320 to 400 nm (UVA), hydrosoluble filters capable of absorbing UV from 280 to 320 nm (UVB) and mixtures thereof;

a first thickening agent chosen from among carrageenans; and

at least one second thickening agent chosen from among synthetic anionic thickening polymers, branched carboxylated thickening polysaccharides and mixtures thereof.

“Physiologically acceptable medium” means a medium compatible with keratin materials. “Keratin materials” refers to the skin and/or lips and/or hair.

Due to the presence of the association of thickening agents according to the invention, the compositions according to the invention are gels.

These compositions are stable and homogeneous. “Homogeneous” means that the composition is in the form of a single phase, without grains visible to the naked eye in suspension in the composition or on the walls of the receptacle, and/or has a uniform texture, i.e. without any gel portions in solution.

Preferably, the composition according to this invention is transparent or translucent. According to some preferred embodiments, the composition according to the invention is a transparent composition. The term transparent or translucent composition as used in the context of this invention means a turbidity value of less than 1000 NTU, preferably less than 800 NTU, preferably less than 500 NTU, and preferably less than 200 NTU, and preferably less than 150 NTU, and preferably less than 90 NTU, and preferably less than 80 NTU. Preferably, the turbidity of compositions is equal to at least 1 NTU. NTUs (nephelometric turbidity units) are units for measurement of the turbidity of a composition. The turbidity measurement is made, for example, with a model 2100P turbidity meter made by the Hach Company, the tubes used for the measurement being references AR397A cat 24347-06. The measurements are made at ambient temperature (from 20° C. to 25° C.).

Preferably, the composition is transparent after application on the skin, i.e. there is no white mark after spreading the composition on the skin.

Another purpose of this invention is a non-therapeutic method of cosmetic treatment of keratin fibers, preferably the skin, comprising application of a composition according to the invention on said keratin fibers.

Hydrosoluble Filters

The composition according to the invention comprises at least one filter chosen from among hydrosoluble filters capable of absorbing UV from 320 to 400 nm (UVA), hydrosoluble filters capable of absorbing UV from 280 to 320 nm (UVB) and mixtures thereof.

The term “hydrosoluble UV filter” refers to any inorganic or organic filter that can be completely dissolved in molecular form in an aqueous liquid phase, or be dissolved in colloidal form (for example in micellar form) in an aqueous liquid phase.

According to one embodiment, the composition comprises at least one hydrosoluble filter capable of absorbing UVA.

According to another embodiment, the composition comprises at least one hydrosoluble filter capable of absorbing UVB.

According to another embodiment, the composition comprises at least one hydrosoluble filter capable of absorbing UVB and UVA

According to another embodiment, the composition comprises a mixture of at least one hydrosoluble filter capable of absorbing UVA with at least one hydrosoluble filter capable of absorbing UVB.

Hydrosoluble filters capable of absorbing UV from 320 to 400 nm (UVA)

Among hydrosoluble filters capable of absorbing UVA, mention may be made of:

Terephthalylidene Dicamphor Sulfonic Acid made by CHIMEX under the name “MEXORYL SX”;

bis-benzoazolyl derivatives as described in EP 669 323, and U.S. Pat. No. 2,463,264 and more specifically the Disodium Phenyl Dibenzimidazole Tetra-sulfonate compound sold under the trade name “NEO HELIOPAN AP” by Haarmann and REIMER.

Preferably, the hydrosoluble capable of absorbing UVA is terephthalylidene dicamphor sulfonic acid.

Hydrosoluble filters capable of absorbing UV from 280 to 320 nm (UVB) Among hydrosoluble filters capable of absorbing UVB, mention may be made of:

derivatives of p-aminobenzoic acid (PABA), such as: PABA,

Glyceryl PABA, and

PEG-25 PABA sold under the name “UVINUL P25” by BASF.

Phenylbenzimidazole Sulfonic Acid particularly sold particularly under the trade name “EUSOLEX 232” by MERCK,

Ferulic acid,

Salicylic acid,

Diethanolamine (DEA) methoxycinnamate,

Benzylidene Camphor Sulfonic Acid manufactured under the name “MEXORYL SL” by CHIMEX, and

Camphor Benzalkonium Methosulfate manufactured under the name “MEXORYL SO” by CHIMEX.

Preferably, the hydrosoluble filter capable of absorbing UVB is Phenylbenzimidazole Sulfonic acid.

Combined hydrosoluble UVA and UVB filters

The composition according to the invention comprises at least one mixed hydrosoluble filter capable of absorbing UVA and UVB, such as a derivative of benzophenone comprising at least one sulfonic radical, such as particularly:

Benzophenone-4 sold under the trade name “UVINUL MS 40” by BASF,

Benzophenone-5, and

Benzophenone-9.

Preferably, the composition according to the invention comprises a total quantity of hydrosoluble filters equal to between 0.2% and 40% by weight, preferably between 0.5% and 40% by weight relative to the total weight of composition, preferably between 1% and 30% by weight, and preferably between 3% and 20% by weight.

Preferably, when the composition comprises at least one hydrosoluble filter capable of absorbing UVA, said filter is present in a quantity equal to between 0.2% and 40% by weight, preferably between 0.5% and 30% by weight relative to the total weight of composition, preferably between 1% and 20% by weight, and preferably between 1.5% and 10% by weight.

Preferably, when the composition comprises at least one hydrosoluble filter capable of absorbing UVB, said filter is present in a quantity equal to between 0.2% and 40% by weight, preferably between 0.5% and 30% by weight relative to the total weight of composition, preferably between 1% and 20% by weight, and preferably between 1.5% and 10% by weight.

When the hydrosoluble UV filter is of the anionic type, for example sulfonic or carboxylic acid, it is preferably associated with an inorganic or organic base such as an alkanolamine.

The term “alkanolamine” means a C2-C10 compound comprising at least one primary, secondary or tertiary amine function, and at least one alcohol function, general primary. Among appropriate alkanolamines, mention may be made of tromethamine and triethanolamine.

Thickening Agent

The composition according to the invention comprises:

a first thickening agent chosen from among carrageenans; and

at least one second thickening agent chosen from among synthetic anionic thickening polymers, branched carboxylated thickening polysaccharides and mixtures thereof.

The second thickening agent according to the invention is polymeric.

In the framework of this application, a thickening agent is an agent capable of increasing by at least 10 times, preferably at least 15 times, and preferably at least 20 times, the viscosity of a mixture comprising 1% by weight of said agent, 0.9% by weight of triethanolamine and water at a temperature of 25° C.

According to one embodiment, the composition according to the invention contains:

a first thickening agent chosen from among carrageenans; and

a second thickening agent chosen from among synthetic anionic thickening polymers.

According to another embodiment, the composition according to the invention contains:

a first thickening agent chosen from among carrageenans; and

at least one second thickening agent chosen from among branched carboxylated thickening polysaccharides.

Preferably, the composition according to the invention comprises:

a first thickening agent chosen from among carrageenans; and

a mixture of at least one synthetic anionic thickening polymer and at least one branched carboxylated thickening polysaccharide.

Carrageenans

The composition according to the invention comprises a first thickening agent chosen from among carrageenans.

Carrageenans are sulfated thickening polysaccharides without any rhamnose.

They make up the cell walls of various red algae (Rhodophycea) belonging to the Gigartinaceae, Hypneaceae, Furcellariaceae and Polyideaceae families. They comprise long anionic polyelectrolyte galactan chains. Their molecular mass can be more than 10⁶. These linear polymers, formed by disaccharide patterns, are composed of two D-galactopyranose units bonded alternately by α- and β-bonds. They are highly sulfated polysaccharides (20-50%) and the a-D-galactopyranosyl residues can be in 3′,6′-anhydro form.

Initially, carrageenans were subdivided into two families depending on their solubility in potassium chloride (KCI). The fractions soluble in KCI were designated by

“Kappa” prefixes while the “Lambda” terms were reserved for insoluble fractions. Later on, the classifications were based on the number and position of sulfate groups and the presence of the 3′,6′-anhydro bridge on 6-D-galactopyranosyl residues. This led to the four main families: κ, λ, β and ω.

The different types of carrageenans do not exist in the pure state, but in the form of hybrids. Thus in the natural state, K and i-carrageenans are in a Kappa-iota hybrid form but one of the two structures may be predominant over the other. The κ-i hybrid state of a structure can be elucidated using specific enzymes, that enrich or diminish the content of one of the two forms. Carrageenans can coexist with their precursors. Carrageenans belonging to different families can coexist in a hybrid structure.

The composition according to the invention preferably comprises at least one lambda type carrageenan. The lambda type carrageenan may or may not be chemically modified. Preferably, the lambda type carrageenan is not chemically modified.

Preferably, the molecular weight (PM) of the carrageenan is between 100,000 and 1,000,000, and more preferably between 250,000 and 800,000. As an example of a lambda type carrageenan, mention may be made of SATIAGUM UTC 10 or SATIAGUM VPC 410 made by DEGUSSA and WELGEENAN ED 1039 made by EUROGUM.

The concentration of carrageenans used in the compositions according to this invention typically varies between 0.01 and 20%, preferably between 0.02 and 10%, preferably between 0.03 and 8%,preferably between 0.05 and 5% and even more preferably between 0.1 and 3% by weight relative to the total weight of the composition.

The composition according to the invention also comprises at least one second thickening agent chosen from among synthetic anionic thickening polymers, branched carboxylated thickening polysaccharides and mixtures thereof.

Synthetic Anionic Thickening Polymers

The composition according to the invention may comprise at least one synthetic anionic thickening polymer as a second thickening agent.

Among examples of synthetic anionic thickening polymers that can be used, mention can be made of cross-linked homopolymers of acrylic acid.

Among cross-linked homopolymers of acrylic acid, mention can be made of those cross-linked by an allyl alcohol ether in the sugar series, for example such as those sold under the names CARBOPOL 980, 981, 954, 2984 and 5984 by NOVEON or products sold under the names SYNTHALEN M and SYNTHALEN K by 3 VSA.

Such acrylic homopolymers can be present in the composition in particulate or non-particulate form. When they are present in particulate form, their average size in the hydrated state is preferably less than or equal to 10 μm, and even more preferably less than or equal to 5 μm. Their average size in the dry or non-hydrated state is preferably less than or equal to 2 μm, and more preferably less than or equal to 1 μm.

Preferably, the acrylic acid homopolymer is present in non-particulate form.

Preferably, an acrylic acid homopolymer that is at least partially neutralized is used The homopolymer used in this invention is chosen particularly among sodium polyacrylates and potassium polyacrylates. Sodium polyacrylate is used in preference.

Acrylic polymers that are neutralized before their use include for example:

sodium polyacrylates such as those marketed under the trade name Cosmedia SP® containing 90% of dry material and 10% of water, or Cosmedia SPL® in inverse emulsion containing about 60% of dry active material, an oil (hydrogenated polydecene) and a surfactant (PPG-5 Laureth-5), both sold by the Cognis company;

partially neutralized sodium polyacrylates, particularly in the form of an inverse emulsion comprising at least one polar oil, for example that sold under the trade name Luvigel® EM by the BASF company; and

mixtures thereof.

An acrylic acid homopolymer that has not been previously neutralized can also be used, that is then partially or completely neutralized by any appropriate means and particularly by the addition of any base such as soda, potash, an alkanolamine such as triethanolamine. This results for example in sodium polyacrylates. Potassium polyacrylates are also suitable for this invention.

Preferably, the composition according to the invention comprises a second thickening agent chosen from among cross-linked homopolymers of acrylic acid, preferably those cross-linked by an allyl alcohol ether in the sugar series.

The concentration of synthetic anionic thickening polymer, preferably a cross-linked homopolymer of acrylic acid (i.e. active material), generally ranges from 0.05 to 40% by weight relative to the total weight of the composition, and preferably from 0.1 to 20% by weight, preferably from 0.2 to 10% by weight, and preferably from 0.25 to 5% by weight, and even more particularly from 0.4 to 3% by weight.

Thickening Branched Carboxylated Polysaccharides

The composition according to the invention may comprise at least one thickening branched carboxylated polysaccharide as a second thickening agent.

Among branched carboxylated thickening polysaccharides, mention may be made in particular of anionic branched polysaccharides based on glucose, mannose, acetylated mannose, pyruvic acid and glucuronic acid. Such a polysaccharide is preferably xanthan gum.

Xanthan is a heteropolysaccharide produced on the industrial scale by aerobic fermentation of the Xanthomonas campestris bacteria. Its structure is composed of a principal chain of β-D-glucoses bonded in β(1,4), similar to cellulose. One glucose molecule out of two carries a lateral trisaccharidic chain composed of an α-D-mannose, a β-glucuronic acid and a terminal β-D-mannose. The internal residue of mannose is generally acetylated on carbon 6. About 30% of terminal mannose residues carry a pyruvate group bonded in chelated form between carbons 4 and 6. The charged glucuronic acids and pyruvic acids can be ionized, and are therefore responsible for the anionic nature of xanthan (negative charge up to pH equal to 1). The content of pyruvate and acetate residues varies depending on the strain of bacteria, the fermentation process, conditions after fermentation and purification steps. These groups can be neutralized in commercial products with Na+, K+ or Ca2+ ions (SATIA, 1986). The neutralized form can be converted into acid form by ion exchange or by dialysis of an acid solution.

Xanthan gums have a molecular weight between 1,000,000 g/mol and 50,000,000 and a viscosity between 0.6 and 1.65 Pa·s for an aqueous composition containing 1% of xanthan gum (measured at 25° C. with a Brookfield LVT type viscometer at 60 rpm). Xanthan gums are represented for example by the products sold under the names Rhodicare® by RHODIA CHIMIE, under the name SATIAXANETM by Cargill Texturizing Solutions (for the food, cosmetic and pharmaceutical industry), under the name NOVAXAN™ by ADM, and under the names Kelzan® and Keltrol® by CP-Kelco.

Preferably, the concentration of branched carboxylated thickening polysaccaride, preferably of xanthan gum (i.e. in active material) generally ranges from 0.01 to 20% by weight relative to the total weight of the composition, and preferably from 0.02 to 10% by weight, and preferably from 0.05 to 5% by weight and even more particularly from 0.1 to 3% by weight.

According to one preferred embodiment, the composition according to the invention comprises at least one carrageenan, and at least one second thickening agent chosen from among cross-linked homopolymers of acrylic acid, xanthan gum and mixtures thereof.

According to one preferred embodiment, the composition according to the invention comprises at least one carrageenan, at least one cross-linked homopolymer of acrylic acid and at least one xanthan gum.

Aqueous Phase

The composition according to the invention comprises a physiologically acceptable aqueous medium.

Preferably, the composition according to the invention comprises an aqueous medium comprising at least water.

The aqueous medium can comprise at least one other organic solvent soluble in water, at 25° C., chosen for example from among:

C1-C4 monoalkanols. The term “C1-C4 monoalkanol” means any saturated, linear or branched alkane compound with 1 to 4 carbon atoms and a single hydroxyl (OH) function. C1-C4 monoalkanols present in compositions according to the invention can be chosen from among methanol, ethanol, propanol, isopropanol, butanol or mixtures thereof. In particular, the choice will be ethanol;

polyols particularly with 2 to 20 carbon atoms, preferably 2 to 6 carbon atoms, such as glycerol, diglycerol, propyleneglycol, isoprene glycol, dipropyleneglycol, butylene glycol, hexylene glycol, 1,3-propanediol, pentylene glycol, simple sugars, hydrosoluble polyalkyleneglycols; and

mixtures thereof.

Preferably, the composition according to the invention comprises at least one C1-C4 monoalkanol, and more particularly ethanol.

They are generally present in concentrations ranging from 0.2 to 90% by weight, more preferably from 0.5 to 50%, and preferably from 1% to 10% by weight, relative to the total weight of the composition.

Preferably, the composition according to the invention also comprises at least one polyol, particularly as defined hereinabove. In particular, the presence of at least one polyol can improve cosmetic properties such as slip during application and reduces stickiness. Preferably, it comprises at least 1% by weight of polyol(s) relative to the total weight of the composition, preferably at least 5% by weight of polyol(s) relative to the total weight of the composition, preferably from 10% to 50% by weight, preferably from 15% to 40% by weight, preferably from 18 to 35% by weight and even more preferably from 20% to 30% by weight.

Preferably, the composition according to the invention comprises a polyol, preferably glycerine, butylene glycol, propylene glycol or dipropylene glycol.

The composition comprises preferably 20 to 90% by weight of water relative to the total weight of the composition, preferably from 30 to 90% by weight, preferably from 25 to 80% by weight and preferably from 40 to 70% by weight.

Acid and/or Base

According to one embodiment, the cosmetic composition according to the invention can comprise an acid and/or a base.

Preferably, the composition according to the invention has a pH of between 4 and 10, preferably between 6 and 9, and preferentially between 6.5 and 8.

Viscosity Compositions according to the invention preferably have a viscosity between 20 and 20000 centipoises, preferably between 20 and 15000 centipoises, preferably between 90 and 1000 centipoises, preferably between 110 and 600 centipoises (1 centipoise=1 mPa·s), measured at 25° C. with a Rheomat RM100® viscosity meter made by Lamy Rheology.

According to one particular embodiment, the composition according to the invention further comprises cosmetically acceptable agents and/or excipients.

The term “cosmetically acceptable” means compatible with the skin and/or integuments thereof, having a pleasant color, odor and texture and not giving rise to unacceptable discomfort (tingling, tightness, redness), liable to dissuade the consumer from using the composition.

Compositions according to the invention can be in the form of an aqueous gel, but also in the form of a gelified emulsion, particularly an oil-in-water emulsion. Preferably, the composition according to the invention is a gel.

When the composition according to the invention is in an oil-in-water emulsion form, it comprises an oily phase dispersed in an aqueous phase. In this case, the aqueous phase comprises hydrosoluble filters and the specific thickening agent according to the invention. The term “oily phase” as used for the invention refers to a phase that is liquid at 20-25° C. and at a pressure of 1.01325 10⁵ Pa. Such an “oily phase” generally comprises at least one oil. The term “oil” denotes any fatty substance that is in liquid form at ambient temperature (20 to 25° C.) and at atmospheric pressure (760 mm of Hg). The “fatty substance” comprises at least one “fatty” hydrocarbon chain, in other words a linear hydrocarbon chain with at least 4 carbon atoms, unsaturated or not unsaturated, possible substituted and in particular a linear C5-C30 hydrocarbon chain. The oily phase may comprise at least one oil and/or at least one liposoluble UV filter.

Preferably, the composition according to the invention does not comprise an oily phase. Preferably, the composition according to the invention comprises less than 5% by weight of an oily phase, in weight of the total weight of the composition, preferably less than 3% by weight, preferably less than 1% by weight. Preferably, compositions according to the invention are oil-free. For the purposes of the invention, the term “oil free” denotes a composition comprising a single liquid phase that is an aqueous phase (a liquid phase containing water). The term “oil” does not cover, for example, hydrosoluble active ingredients, UV filters soluble in water and glycols soluble in water.

Finally, another purpose of this invention is a non-therapeutic method of cosmetic treatment of keratin fibers, preferably the skin, comprising application of a composition according to the invention on said keratin fibers. Such a method is intended particularly to protect keratin fibers, and particularly the skin, against UV radiation.

Preferably, the composition according to the invention comprises less than 5% by weight of oil soluble UV filters, in weight of the total weight of the composition, preferably less than 3% by weight, preferably less than 1% by weight. Preferably, compositions according to the invention do not comprise any oil soluble UV filter.

Preferably, the compositions according to the invention are in the form of an aqueous gel, and comprise, preferably consist in, a physiologically acceptable aqueous medium, at least one hydrosoluble filter capable of absorbing UV from 320 to 400 nm (UVA); at least one hydrosoluble filter capable of absorbing UV from 280 to 320 nm (UVB); a first thickening agent chosen from among carrageenans; at least one second thickening agent chosen from among synthetic anionic thickening polymers, branched carboxylated thickening polysaccharides and mixtures thereof; and optionally an acid and/or a base.

We will now give concrete examples illustrating the invention, but that are in no way restrictive.

In the examples, the temperature is ambient temperature (20° C.) expressed in degrees Celsius unless mentioned otherwise, and the pressure is atmospheric pressure, unless mentioned otherwise. In the examples, quantities of the ingredients of the compositions are given as a by weight relative to the total weight of the composition.

### EXAMPLE 1

### Preparation and Evaluation of Compositions According to the Invention and Comparative Compositions

Compositions A to D in Table 1 below were prepared as follows. Compositions B and D are compositions according to the invention. Compositions A and C are comparative composition. Comparative compositions are different from compositions according to the invention by the nature of the thickening agent:

Comparative composition A contains only a carbomer as thickening agent.

Comparative composition C comprises xanthan gum and a carbomer.

In the following tables, the expression “m.a.” means “active material”.

Preparation of Compositions

Preservation agents and water are mixed in a beaker.

Ingredients of the “aqueous filter phase”, namely water, hydrosoluble UV filters, TEA and tromethamine, are mixed while stirring a clear mixture. Once this filter phase is ready, the preservation agents and water are added into the mixture.

The carbomer (when it is present) is dissolved in water under a deflocculator (at about 600 rpm). Once it has been dissolved, it is neutralized using TEA. Xanthan gum (when it is present) is added, then wait for it to stabilize. Similarly, the carrageenan (when it is present) is added, then wait for it to stabilize.

The viscosity and turbidity of compositions are evaluated as described in the description.

The results are given in Table 1.

TABLE 1

| | | A | B | C | D |
|---|---|---|---|---|---|
| | | (comparative) | (invention) | (comparative) | (invention) |
| Gel | Water | Qs 100 | | | |
| phase | Preservatives | QS | | | |
| | CARBOMER | 0.5 | 0.5 | 0.5 | 0.5 |
| | (carbopol 980) | | | | |
| | Triethanolamine | 0.7 | 0.7 | 0.7 | 0.7 |
| | (TEA) | | | | |
| | Xanthan gum | — | — | 0.5 | 0.5 |
| | (KELTROL CG-T | | | | |
| | CP KELCO) | | | | |
| | CARRAGEENAN | — | 0.3 | — | 0.3 |
| | (SATIAGUM VPC | | | | |
| | 410 CARGILL) | | | | |
| Aqueous | Water | 15 | 15 | 15 | 15 |
| filter | Phenylbenzimidazole | 8 | 8 | 8 | 8 |
| phase | Sulfonic Acid | | | | |
| | (Eusolex 232 made | | | | |
| | by Merck) | | | | |
| | Terephthalylidene | 10 | 10 | 10 | 10 |
| | Dicamphor | (3.3% m.a.) | (3.3% m.a.) | (3.3% m.a.) | (3.3% m.a.) |
| | Sulfonic Acid | | | | |
| | (Mexoryl SX made by | | | | |
| | Chimex; 33% in m.a.) | | | | |
| | TEA | 2.5 | 2.5 | 2.5 | 2.5 |
| | Tromethamine | 3.5 | 3.5 | 3.5 | 3.5 |

| | | | | |
|---|---|---|---|---|
| Viscosity | 130 | 115 | 407 | 558 |
| (in centipoises) | | | | |
| Turbidity | 90 | 60 | 92 | 52 |
| (in NTU) | | | | |

The results show that:

with comparative formula A: the formula has a certain viscosity but no turbidity of less than 80 NTU;

with the formula according to the invention B: the formula has a certain viscosity and is transparent;

with comparative formula C: the formula has a certain viscosity but no turbidity of less than 80 NTU; and

with the formula according to the invention D: the formula is transparent and has no loss of viscosity.

In conclusion, it is found that the formulas according to the invention B and D are the formulas that have the most useful viscosities, while remaining transparent (low turbidities).

### EXAMPLE 2

### Evaluation of the Transparency of a Composition According to the Invention

The composition F1 in Table 2 below was prepared as indicated in example 1. Formula F1 includes the association of thickening agents according to the invention.

The viscosity and turbidity of the composition are evaluated as described in the description. The results are given in Table 2.

TABLE 2

| | | Formula F1 |
|---|---|---|
| | | (invention) |
| Gel phase | Water | 59.5 |
| | Preservatives | 0.5 |
| | CARBOMER | 0.5 |
| | (carbopol 980) | |
| | Triethanolamine (TEA) | 0.7 |
| | Xanthan gum | 0.3 |
| | (KELTROL CG-T | |
| | CP KELCO) | |
| | CARRAGEENAN | 0.5 |
| | (SATIAGUM VPC 410 | |
| | CARGILL) | |
| Aqueous | Water | 15 |
| filter | Terephthalylidene | 10 |
| phase | Dicamphor | (3.3% m.a.) |
| | Sulfonic Acid | |
| | (Mexoryl SX made by | |
| | Chimex; 33% in m.a.) | |
| | TEA | 2.5 |
| | Tromethamine | 3.5 |

| | |
|---|---|
| Turbidity (in NTU) | 52 |

It is found that formula F1 according to the invention is transparent.

### EXAMPLE 3

### Preparation and Evaluation of Compositions According to the Invention

Compositions F2 and F3 according to the invention (see Table 3), are prepared as described in example 1.

Their homogeneity and transparency, and their stickiness and slip during application, are evaluated.

TABLE 3

| | | F2 | F3 |
|---|---|---|---|
| Gel | Water | Qs 100 | |
| phase | Preservatives | QS | |
| | CARBOMER | 0.5 | 0.5 |
| | (carbopol 980) | | |
| | Triethanolamine (TEA) | 0.7 | 0.7 |
| | Xanthan gum | 0.3 | 0.3 |
| | (KELTROL CG-T | | |
| | CP KELCO) | | |
| | CARRAGEENAN | 0.5 | 0.5 |
| | (SATIAGUM VPC410 | | |
| | CARGILL) | | |
| Aqueous | Water | 15 | 15 |
| filter | Phenylbenzimidazole | 8 | 8 |
| phase | Sulfonic Acid | | |
| | (Eusolex 232 made by Merck) | | |
| | Terephthalylidene Dicamphor | 10 | 10 |
| | Sulfonic Acid | (3.3% m.a.) | (3.3% m.a.) |
| | (Mexoryl SX made by | | |
| | Chimex; 33% in m.a.) | | |
| | TEA | 2.5 | 2.5 |
| | Tromethamine | 3.5 | 3.5 |
| | Glycerin | — | 2 |
| | Propylene glycol | — | 19 |
| | Butylene glycol | — | 6.5 |

| | | |
|---|---|---|
| Homogeneity | Homogeneous | Homogeneous |
| Turbidity (in NTU) | 71 | 50 |

Compositions according to the invention are homogeneous and transparent. Furthermore, the presence of polyols as in composition F3, reduces stickiness and improves slip during application.

1. A composition comprising, in a physiologically acceptable aqueous medium:
at least one filter chosen from among hydrosoluble filters capable of absorbing UV from 320 to 400 nm (UVA), hydrosoluble filters capable of absorbing UV from 280 to 320 nm (UVB) and mixtures thereof;
a first thickening agent chosen from among carrageenans; and
at least one second thickening agent chosen from among synthetic anionic thickening polymers, branched carboxylated thickening polysaccharides and mixtures thereof.

2. The composition according to claim 1, wherein the hydrosoluble filter capable of absorbing UVA is chosen from among terephthalylidene dicamphor sulfonic acid, bis-benzoazolyl derivatives.

3. The composition according to claim 1, wherein the hydrosoluble filter capable of absorbing UVB is chosen from among PABA, glyceryl PABA, PEG-25 PABA, Phenylbenzimidazole Sulfonic acid, ferulic acid, salicylic acid, DEA methoxycinnamate, Benzylidene Camphor Sulfonic acid and Camphor Benzalkonium Methosulfate.

4. The composition according to claim 1, which comprises a total quantity of hydrosoluble filters equal to between 0.2% and 40% by weight.

5. The composition according to claim 1, which comprises at least one C1-C4 monoalkanol, and/or at least one polyol.

6. The composition according to claim 5 which comprises at least one C1-C4 monoalkanol, in a quantity varying from 0.2 to 90% by weight and/or at least one polyol in a quantity of at least 1% by weight relative to the total weight of the composition.

7. The composition according to claim 1 wherein the first thickening agent is a lambda type carrageenan.

8. The composition according to claim 1 wherein the concentration of carrageenans varies between 0.01 and 20% relative to the total weight of the composition.

9. The composition according to claim 1 wherein, the synthetic anionic thickening polymer is chosen from among cross-linked homopolymers of acrylic acid.

10. The composition according to claim 1 wherein the synthetic anionic thickening polymer is present as active material in a quantity varying from 0.05 to 40% by weight relative to the total weight of the composition.

11. The composition according to claim 1 wherein the branched carboxylated thickening polysaccaride is chosen from among anionic branched polysaccharides based on glucose, mannose, acetylated mannose, pyruvic acid and glucuronic acid.

12. The composition according to claim 1, wherein the branched carboxylated thickening polysaccharide is present as active material in a quantity varying from 0.01 to 20% by weight relative to the total weight of the composition.

13. The composition according to claim 1, which comprises at least one carrageenan, and at least one second thickening agent chosen from among cross-linked homopolymers of acrylic acid, xanthan gum and mixtures thereof.

14. The composition according to claim 1, which comprises at least one carrageenan, at least one cross-linked homopolymer of acrylic acid and at least one xanthan gum.

15. The composition according to claim 1, which comprises less than 5% by weight of an oily phase, in weight of the total weight of the composition and/or it comprises less than 5% by weight of oil soluble UV filters, in weight of the total weight of the composition.

16. The composition according to claim 2, wherein the hydrosoluble filter capable of absorbing UVB is chosen from among PABA, glyceryl PABA, PEG-25 PABA, Phenylbenzimidazole Sulfonic acid, ferulic acid, salicylic acid, DEA methoxycinnamate, Benzylidene Camphor Sulfonic acid and Camphor Benzalkonium Methosulfate.

17. The composition according to claim 2, which comprises a total quantity of hydrosoluble filters equal to between 0.2% and 40% by weight.

18. The composition according to claim 3, which comprises a total quantity of hydrosoluble filters equal to between 0.2% and 40% by weight.

19. The composition according to claim 2, which comprises at least one C1-C4 monoalkanol and/or at least one polyol.

20. The composition according to claim 3, which comprises at least one C1-C4 monoalkanol and/or at least one polyol.
